(19) United States
(12) Patent Application Publication
(10) Pub. No.: US 2024/0156799 A1
(43) Pub. Date: May 16, 2024

(54) USES OF BUPIVACAINE MULTIVESICULAR LIPOSOMES AS STELLATE GANGLION BLOCK FOR TREATING ANXIETY DISORDERS AND TRAUMATIC BRAIN INJURY

(71) Applicant: Pacira Pharmaceuticals, Inc., San Diego, CA (US)

(72) Inventors: John Spranger, Hingham, MA (US); Roy Winston, Tampa, FL (US); Jonathan Slonin, Palm City, FL (US)

(21) Appl. No.: 18/503,474

(22) Filed: Nov. 7, 2023

Related U.S. Application Data
(60) Provisional application No. 63/505,979, filed on Jun. 2, 2023.
(60) Provisional application No. 63/383,177, filed on Nov. 10, 2022.

(51) Int. Cl. A61K 31/445; A61K 9/127; A61K 31/135; A61P 25/00

(52) U.S. Cl. CPC A61K 31/445; A61K 9/127; A61K 31/135; A61P 25/00

(57) ABSTRACT

Embodiments of the present disclosure relate to methods of using bupivacaine multivesicular liposomes as stellate ganglion block for treating or ameliorating an anxiety disorder or a traumatic brain injury associated with overactive or unbalanced sympathetic nervous system, including but not limited to, generalized anxiety disorders, panic disorder, and post-traumatic stress disorder.

### INCORPORATION BY REFERENCE PRIORITY APPLICATIONS

The present application claims the benefit of priority to U.S. Provisional Application Nos. 63/383,177, filed Nov. 10, 2022 and 63/505,979, filed Jun. 2, 2023, which are incorporated by reference in their entirety.

### BACKGROUND

### Field

The present disclosure relates to compositions of bupivacaine multivesicular liposomes (MVLs) and uses thereof as stellate ganglion block for treating anxiety disorders and traumatic brain injury.

### Description of the Related Art

The stellate ganglion is formed by a fusion of the C7, C8, and T1 regions (for example, the C7, C8, and T1 ganglia) of the paravertebral chain. Bupivacaine is a versatile drug that has been shown to be efficacious for a wide variety of indications, including: postoperative analgesia, peripheral nerve blocking, diagnostic and therapeutic procedures, and obstetrical procedures. See Obaid Malik, et al., Emerging roles of liposomal bupivacaine in anesthesia practice, 33 Journal of Anaesthesiology Clinical Pharmacology, 151 (2017).

The lifetime prevalence of posttraumatic stress disorder (PTSD) is estimated to range between 13% and 20% for women, and 6%-8% for men. See R. A. Bryant, Post-traumatic stress disorder: a state-of-the-art review of evidence and challenges, World Psychiatry 18 (2019). Both psychological and neuropathological factors underpin PTSD. Psychologically, the most theory implicates fear conditioning, wherein a surge in stress hormones released in the traumatic period results in strong associative learning between cues present at the time of trauma and fear responses. Similar subsequent cues are implicitly assumed to confer immediate threats, leading to a re-experiencing of fear and associated physiological changes (e.g., sympathetic nervous activation). This model also posits that recovery from PTSD involves extinction learning. Id. Pathophysiological alterations also occur in PTSD that may be influenced by SGB and ketamine. These include sympathetic dysregulation, functional central nervous system (CNS) plasticity, and structural changes that include changes in grey matter volumes in areas associated with emotions. Id.

Traumatic brain injury (TBI) commonly occurs with PTSD. See C. W. Chan, et al., A case of sympathetically mediated headache treated with stellate ganglion blockade, Pain Med. 11 (2010). For example, approximately 48% of patients with mild TBI suffer from concomitant PTSD. See D. E. Nampiaparampil, Prevalence of chronic pain after traumatic brain injury: a systematic review, JAMA, 13 (2008). Neuropathologically, TBI may result in direct injury to neuronal structures leading to excitotoxicity, apoptosis and decreased neurogenesis, inflammation, demyelination, white and gray matter pathology, and disruption of the blood-brain barrier, all of which confer increased long-term risk of neurodegenerative conditions; cognitive and behavioral changes including depression and post-traumatic stress disorder; and a markedly increased risk of disability. See D. Pavlovic, et al., Traumatic brain injury: neuropathological, neurocognitive and neurobehavioral sequelae, Pituitary, 22 (2019); see also, H. M. Bramlett, et al., Long-Term Consequences of Traumatic Brain Injury: Current Status of Potential Mechanisms of Injury and Neurological Outcomes, J. Neurotrauma, 32 (2015).

### SUMMARY

Some embodiments of the present disclosure relate to a method of treating a patient who has suffered from or is at risk of suffering from an anxiety disorder or a traumatic brain injury (TBI), including: administering a composition comprising an effective amount of bupivacaine multivesicular liposomes (MVLs) to one or more nerves of the stellate ganglion of the patient, or an autonomic tissue area peripheral to the stellate ganglion or both. In some embodiments, the method further comprises identifying or selecting a patient who has suffered from or is at risk of suffering from the anxiety disorder or the TBI.

Some embodiments of the present disclosure relate to a method of treating or ameliorating an anxiety disorder or a traumatic brain injury associated with an overactive or unbalanced sympathetic nervous system in a patient in need thereof, including: administering a composition comprising an effective amount of bupivacaine MVLs to one or more nerves of the stellate ganglion of the patient, or an autonomic tissue area peripheral to the stellate ganglion, or both. In some embodiments, the method further comprises identifying or selecting a patient who has suffered from or is at risk of suffering from the overactive or unbalanced sympathetic nervous system.

Some embodiments of the present disclosure relate to a method of reducing or interrupting sympathetic stimulation to one or more brain regions associated with an anxiety disorder or a traumatic brain injury in a patient in need thereof, including: administering a composition comprising an effective amount of bupivacaine MVLs to one or more nerves of the stellate ganglion of the patient, or an autonomic tissue area peripheral to the stellate ganglion, or both. In some embodiments, the method further comprises identifying or selecting a patient who has suffered from or is at risk of suffering from overactive or overstimulated sympathetic nervous system.

In any embodiments of the methods described herein, the anxiety disorder can be a generalized anxiety disorder, panic disorder, post-traumatic stress disorder (PTSD), a phobia related disorder, or obsessive-compulsive disorder. In some embodiments, the anxiety disorder is PTSD. In some embodiments, the method results in temporarily interruption of sympathetic stimulation to one or more brain regions associated with the anxiety disorder. In some embodiments, the patient who suffered from or is at risk of suffering from PTSD has also suffered from or is at risk of suffering from a traumatic brain injury. In some embodiments, the patient who suffered from or is at risk of suffering from a traumatic brain injury has also suffered from or is at risk of suffering from PTSD. In some embodiments, the one or more nerves of the stellate ganglion comprise one or more nerves that provide sympathetic stimulation to one or more brain regions associated with the anxiety disorder.

Some embodiments of the present disclosure relate to a method of treating or ameliorating one or more symptoms associated with a traumatic brain injury (TBI) in a patient in need thereof, including: identifying or selecting a patient who has suffered from a TBI; administering a composition including an effective amount of bupivacaine multivesicular liposomes (MVLs) to one or more nerves of the stellate ganglion of the patient, or an autonomic tissue area peripheral to the stellate ganglion, or both. In some embodiments, the one or more nerves of the stellate ganglion include one or more nerves that provide sympathetic stimulation to one or more brain regions associated with the TBI. In some embodiments, the one or more symptoms of the TBI include headaches, post traumatic headaches, chronic pain, neck pain, upper extremity pain, depression, or anxiety. In some embodiments, the patient also suffers from post-traumatic stress disorder (PTSD).

In some embodiments, the method disclosed herein further incudes measuring or receiving information on a baseline level of one or more proinflammatory biomarkers in the blood prior to the administration of the composition of bupivacaine MVLs. In some embodiments, the one or more inflammatory biomarkers include interleukin-1β (IL-1β), interleukin-6 (IL-6), tumor necrosis factor-α (TNFα), neurofilament light chain (NfL), glial fibrillary acidic protein (GFAP), C-reactive protein, nerve growth factor (NGF) and brain derived neurotrophic factor (BDNF). In some embodiments, the method reduces the level of one or more proinflammatory biomarkers in the blood. In some embodiments, the method further includes administering a sedative to the patient prior to the administration of the composition of bupivacaine MVLs. In certain embodiments, the method further includes co-administering an effective amount of ketamine by intravenous continuous infusion or intravenous bolus injection. In further embodiments, the effective amount of ketamine is from about 0.5 to about 1 mg/kg.

In some embodiments, the method causes a temporary reduction in the ability of the sympathetic nervous system to release norepinephrine. In some embodiments, the one or more nerves of the stellate ganglion comprise one or more nerves that provide sympathetic stimulation to one or more brain regions associated with anxiety disorders.

In some embodiments, the method further includes assessing or receiving baseline information on one or more tests including Posttraumatic Stress Disorder Checklist (PCL-5), Headache Impact Test (HIT-6), Hospital Anxiety and Depression Scale (HADS), Quality of Life after Brain Injury (QoLIBRI), (Sheehan Suicide Tracking Scale (S-STS), Central Sensitization Inventory (CSI), headache frequency, average and worst headache scores (0-10 numerical rating scale (NRS)), and average and worst overall pain (0-10 NRS scale). In some embodiments, the method results in at least a 6-point decrease on HIT-6 or 10-point decrease on PCL-5, optionally combined with a Patients' Global Impression of Change (PGIC score)≥4/7. In some embodiments, the change in HIT-6, PCL-5 or PGIC score is determined 4, 6, or 8 weeks after the administration of the composition of bupivacaine MVLs.

In some embodiments of the methods described herein, the concentration of bupivacaine in the composition is from about 1 mg/mL to about 30 mg/mL, or from about 2 mg/mL to about 20 mg/mL. In one embodiment, the concentration of bupivacaine in the composition is about 13.3 mg/mL. In some embodiments, the effective amount of bupivacaine administered is from about 10 mg to about 200 mg, from about 20 mg to about 100 mg, or from about 25 mg to about 75 mg. In some embodiments of the methods described herein, the total volume of the composition administered is from about 1 mL to about 20 mL. In some embodiments, the total volume of the composition administered is from about 5 mL to about 10 mL for unilateral stellate ganglion block (for example, about 5 mL, 6 mL, 7 mL, 8 mL, 9 mL or 10 mL). In further embodiments, the total volume of the composition administered is from about 10 mL to about 20 mL for bilateral stellate ganglion block. In some embodiments, bupivacaine is in a salt form. In further embodiments, the bupivacaine is in the form of bupivacaine phosphate.

In some embodiments of the methods described herein, the administering temporarily reduces or interrupts sympathetic stimulation to the nervous system. In some embodiments, the temporary reduction or interruption of sympathetic stimulation to the nervous system is for up to 3 days, 7 days, 10 days, 14 days, 21 days, 1, 2, 3, 4, 5 or 6 months.

In any embodiment of the methods described herein, the administrating of the composition of bupivacaine MVLs comprises:
navigating a treatment apparatus to stellate ganglion of the patient, or an autonomic tissue area peripheral to the stellate ganglion, wherein the treatment apparatus comprises a needle and a compartment for the composition of bupivacaine MVLs;
injecting bupivacaine encapsulated MVLs from the treatment apparatus to one or more nerves of the stellate ganglion, or the autonomic tissue area peripheral to the stellate ganglion; and
removing the treatment apparatus from the patient.

In some embodiments, navigating the treatment apparatus to the stellate ganglion comprises inserting the needle of the treatment apparatus percutaneously to one or more nerves of the stellate ganglion area. In some embodiments, navigating the treatment apparatus to the stellate ganglion comprises inserting the needle of the treatment apparatus percutaneously to one or more nerves of the stellate ganglion, or one or more nerves of the autonomic tissue area peripheral to the stellate ganglion, or both. In some embodiments, navigating the treatment apparatus to the stellate ganglion comprises placing the needle above the longus colli muscle. In further embodiments, navigating the treatment apparatus to the stellate ganglion comprises guiding the needle using an imaging guide. In further embodiments, the imaging guide comprises fluoroscopic or ultrasound imaging guide.

In some embodiments of the methods described herein, the injection comprises bolus injection, continuous infusion, or a combination thereof. In some embodiments, the administering is to the left stellate ganglion. In some embodiments, the administering is to the right stellate ganglion. In some embodiments, the administering is to both the left and the right stellate ganglion. In some embodiments, the one or more desired nerves of the stellate ganglion or the autonomic tissue area peripheral to the stellate ganglion comprise one or more desired nerves of the autonomic tissue area peripheral to the stellate ganglion. In some other embodiments, the one or more desired nerves of the stellate ganglion or the autonomic tissue area peripheral to the stellate ganglion comprise one or more desired nerves of the autonomic tissue area peripheral to the stellate ganglion. In some other embodiments, the one or more desired nerves of the stellate ganglion or the autonomic tissue area peripheral to the stellate ganglion comprise one or more desired nerves of a paravertebral chain. In some such embodiments, the one or more desired nerves of the paravertebral chain comprises one or more desired nerves of one or more of a C7 region of the paravertebral chain, a C8 region of the paravertebral chain, a T1 region of the paravertebral chain, or a T2 region of the paravertebral chain, or combinations thereof. In some further embodiments, the one or more desired nerves of the paravertebral chain comprise one or more desired nerves of the T1 region of a paravertebral chain or the T2 region of the paravertebral chain, or a combination thereof. In certain embodiments, the one or more desired nerves of the paravertebral chain comprises one or more desired nerves of one or more of a C6 region of the paravertebral chain, a C7 region of the paravertebral chain, or combinations thereof.

### BRIEF DESCRIPTION OF THE FIGURE

FIG. 1 is a flow diagram summarizing a study to evaluate the efficacious treatments of ketamine and stellate ganglion block (SGB) with bupivacaine infusion, compared to sham therapies, for headaches associated with traumatic brain injury (TBI) and post-traumatic stress disorder (PTSD).

### DETAILED DESCRIPTION

Embodiments of the present disclosure relate to methods of using bupivacaine multivesicular liposomes (MVLs) as stellate ganglion block (SGB) for treating conditions such as anxiety disorders, generalized anxiety disorders, panic disorder, post-traumatic stress disorder (PTSD), phobia related disorder, obsessive-compulsive disorder, and a traumatic brain injury (TBI).

The section headings used herein are for organizational purposes only and are not to be construed as limiting the subject matter described.

### Definitions

The section headings used herein are for organizational purposes only and are not to be construed as limiting the subject matter described.

Unless defined otherwise, all technical and scientific terms used herein have the same meaning as is commonly understood by one of ordinary skill in the art. The use of the term “including” as well as other forms, such as “include”, “includes,” and “included,” is not limiting. The use of the term “having” as well as other forms, such as “have”, “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, formulation, or device, the term “comprising” means that the compound, composition, formulation, or device includes at least the recited features or components, but may also include additional features or components.

As used herein, the terms “bupivacaine encapsulated multivesicular liposomes”, or “bupivacaine MVLs” refer to a multivesicular liposome composition encapsulating bupivacaine. In some embodiments, the composition is a pharmaceutical formulation, where the bupivacaine encapsulated multivesicular liposome particles are suspended in a liquid suspending medium to form a suspension. In some such embodiments, the BUP-MVL suspension may also include free or unencapsulated bupivacaine. In some cases, the free or unencapsulated bupivacaine may be less than about 8%, 7%, 6%, 5%, 4%, 3%, 2%, 1%, 0.5%, 0.2% or 0.1%, by weight of the total amount of the bupivacaine in the composition, or in a range defined by any of the two preceding values. In some embodiment, the free bupivacaine may be about 5% or less by weight of the total amount of the bupivacaine in the composition. In further embodiments, the free bupivacaine may be about 8% or less during the shelf life of the product (i.e., up to 2 years when stored at 2-8° C.).

As used herein, the term “encapsulated” means that bupivacaine is inside a liposomal particle, for example, the MVL particles. In some instances, bupivacaine may also be on an inner surface, or intercalated in a membrane, of the MVLs.

As used herein, the term “unencapsulated bupivacaine” or “free bupivacaine” refers to bupivacaine outside the liposomal particles, for example the MVL particles. For example, unencapsulated bupivacaine may reside in the suspending solution of these particles.

As used herein, “effective amount” or “pharmaceutically effective amount” is meant an amount of therapeutic agent, which has a therapeutic effect. The dosages of a pharmaceutically active ingredient which are useful in treatment are therapeutically effective amounts. Thus, as used herein, a therapeutically effective amount means an amount of therapeutic agent which produces the desired therapeutic effect as judged by clinical trial results and/or model animal studies.

“Treat,” “treatment,” or “treating,” as used herein refers to administering a pharmaceutical composition/formulation for prophylactic and/or therapeutic purposes. The term “prophylactic treatment” refers to treating a patient who is not yet suffering from a disease, but who is susceptible to, or otherwise at risk of, a particular disease, whereby the treatment reduces the likelihood that the patient will develop a disease. The term “therapeutic treatment” refers to administering treatment to a patient already suffering from a disease.

### Bupivacaine Multivesicular Liposomes

Some aspect of the present disclosure relates to a composition of bupivacaine encapsulated multivesicular liposomes (MVLs), comprising: bupivacaine residing inside a plurality of internal aqueous chambers of the MVLs separated by lipid membranes, wherein the lipid membranes comprise 1, 2-dierucoylphosphatidylcholine (DEPC), 1,2-dipalmitoyl-sn-glycero-3 phospho-rac-(1-glycerol) (DPPG), and at least one neutral lipid; and an aqueous medium in which the bupivacaine encapsulated MVLs are suspended. Additional non-limiting description of the composition of bupivacaine encapsulated multivesicular liposomes is included in U.S. Publication No. 2002-0039596 A1 and U.S. Pat. Nos. 6,045,824 and 11,033,495, each of which is incorporated by reference in its entirety.

In some embodiments, the composition of bupivacaine encapsulated MVLs may have a volume of 10 mL or 20 mL for a single dose administration. In some embodiments, the percent packed particle volume (% PPV) of the composition of bupivacaine encapsulated MVLs is about 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64% or 65%. In some such embodiments, the concentration of the bupivacaine in the composition is from about 11 mg/mL to about 17 mg/mL. In one embodiment, the concentration of the bupivacaine in the composition is about 13.3 mg/mL. In other embodiments, the concentration of the bupivacaine in the composition is from about 18 mg/mL to about 22 mg/mL, or about 20 mg/mL. In further embodiments, the composition comprises less than 5%, 4%, 3%, 2% or 1% unencapsulated bupivacaine, wherein the amount of unencapsulated bupivacaine is calculate based on the total weight of the bupivacaine in the composition. In some embodiments, the d₅₀ of the multivesicular liposomes in the composition is about 24 μm to about 31 μm. In one embodiment, the d₅₀ of the multivesicular liposomes in the composition is about 27 μm.

### Pharmaceutical Compositions

In some embodiments, the composition comprising bupivacaine MVLs is a pharmaceutical formulation includes a pharmaceutically acceptable carrier. Effective injectable bupivacaine MVLs compositions is in a liquid suspension form. Such injectable suspension compositions require a liquid suspending medium, with or without adjuvants, as a vehicle. The suspending medium can be, for example, aqueous solutions of sodium chloride (i.e., saline solution), dextrose, sucrose, polyvinylpyrrolidone, polyethylene glycol, a pH modifying agent described herein, or combinations of the above. In some embodiments, the suspending medium of bupivacaine MVLs is a saline solution, optionally contain a tonicity agent such as dextrose and/or a pH modifying agent such as lysine. In other embodiment, the suspending medium of bupivacaine MVLs is a buffered saline solution (e.g., containing a phosphate buffer).

Suitable physiologically acceptable storage solution components are used to keep the compound suspended in suspension compositions. The storage solution components can be chosen from thickeners such as carboxymethylcellulose, polyvinylpyrrolidone, gelatin and the alginates. Many surfactants are also useful as suspending agents. The suspending medium could also contain lecithin, alkylphenol polyethylene oxide adducts, naphthalenesulfonates, alkylbenzenesulfonates, or the polyoxyethylene sorbitan esters. In some embodiments, the bupivacaine MVL composition is free or substantially free of any additive of preservatives.

In any embodiments of the composition of bupivacaine encapsulated MVLs described herein, the composition may be a pharmaceutical composition suitable for human administration. In further embodiments, the composition may be an aqueous suspension of bupivacaine encapsulated MVL particles. In some embodiments, the administered bupivacaine is in a salt form. In further embodiments, the administered bupivacaine is in the form of bupivacaine phosphate.

### Methods of Treatment

Some embodiments of the present disclosure relate to a method of treating a patient who has suffered from or is at risk of suffering from an anxiety disorder or a traumatic brain injury (TBI), comprising administering a composition containing bupivacaine multivesicular liposomes (MVLs) as described herein. Some embodiments of the present disclosure relate to a method of treating or ameliorating an overactive or unbalanced sympathetic nervous system associated anxiety disorder in a patient in need thereof, comprising administering a composition containing bupivacaine MVLs. Certain embodiments of the present disclosure relate to a method of treating or ameliorating an anxiety disorder or a traumatic brain injury associated with an overactive or unbalanced sympathetic nervous system in a patient in need thereof, comprising administering a composition containing bupivacaine MVLs. The present embodiments also provide a method of reducing or interrupting sympathetic stimulation to the nervous system of a patient in need thereof, comprising administering a composition containing bupivacaine MVLs. The present embodiments also provide a method of reducing or interrupting sympathetic stimulation to one or more brain regions associated with an anxiety disorder or a traumatic brain injury in a patient in need thereof, comprising administering a composition containing bupivacaine MVLs. In some embodiments, the methods disclosed herein include administering a composition containing bupivacaine MVLs to one or more nerves of stellate ganglion of the patient, or one or more nerves of an autonomic tissue area peripheral to the stellate ganglion, or a combination thereof. In some embodiments, the methods include identifying or selecting a patient who has suffered from or is at risk of suffering from the anxiety disorder or the TBI. In some embodiments, the methods include identifying or selecting a patient who has suffered from or is at risk of suffering from the overactive or unbalanced sympathetic nervous system. In some embodiments, the methods include identifying or selecting a patient who has suffered from or is at risk of suffering from overactive or overstimulated sympathetic nervous system. In some embodiments of the methods disclosed herein, the administration of the bupivacaine MVLs to one or more nerves of stellate ganglion of the patient, or autonomic tissue area peripheral to the stellate ganglion, or a combination thereof, temporarily reduces or interrupts sympathetic stimulation to the nervous system for a sustained period of time. In some embodiments, the temporary reduction or interruption of sympathetic stimulation to the nervous system is for up to 3 days, 7 days, 10 days, 14 days, 21 days, 1 month, 2 months, 3 months, 4 months, 5 months or 6 months.

Certain embodiments employ methods to at least temporarily interrupt the activity of certain nerves of the stellate ganglion and/or autonomic tissue peripheral to the stellate ganglion. Certain embodiments employ methods to the stellate ganglion and/or autonomic tissue peripheral to the stellate ganglion to perform a stellate ganglion block. The nerves of the stellate ganglion and/or autonomic tissue peripheral to the stellate ganglion are connected to and can cause stimulation of brain regions, such as the amygdala, which are associated with anxiety disorders. Methods of the present disclosure may, for example, prevent the release of one or more neurotransmitters that may cause an undesirable affect. In some embodiments, the methods disclosed herein may prevent the release of neurotransmitters (for example, catecholamines, such as epinephrine and norepinephrine) that may cause anxiety disorders. In some embodiments, the methods disclosed herein may allow for treatment of anxiety disorders for which psychotropics are not effective. In some embodiments, methods of treatment of the nerves of the stellate ganglion and/or autonomic tissue peripheral to the stellate ganglion can be used to treat anxiety disorders such as generalized anxiety disorders, panic disorder, PTSD, phobia related disorders (e.g., social phobia), and obsessive-compulsive disorder.

Certain embodiments also provide a method of treating or ameliorating one or more symptoms associated with a traumatic brain injury (TBI) in a patient in need thereof, comprising identifying or selecting a patient who has suffered from a TBI; and administering a composition containing bupivacaine MVLs. In some embodiments, the one or more nerves of the stellate ganglion comprise one or more nerves that provide sympathetic stimulation to one or more brain regions associated with the TBI. In some embodiments, the one or more symptoms of the TBI comprise headaches, post traumatic headaches, chronic pain, neck pain, upper extremity pain, depression, or anxiety.

In certain embodiments, the methods described herein further comprise measuring or receiving information on a baseline level of one or more proinflammatory biomarkers in the blood prior to the administration of the composition of bupivacaine MVLs. In some embodiments, the one or more inflammatory biomarkers are selected from the group consisting of interleukin-1β (IL-1β), interleukin-6 (IL-6), tumor necrosis factor-α (TNFα), neurofilament light chain (NfL), glial fibrillary acidic protein (GFAP), C-reactive protein, nerve growth factor (NGF) and brain derived neurotrophic factor (BDNF). In certain embodiments, the methods described herein reduce the level of one or more proinflammatory biomarkers in the blood.

In some embodiments, the methods described herein further comprise administering a sedative to the patient prior to the administration of the composition of bupivacaine MVLs. In some embodiments, the sedative comprises midazolam or ketamine, or a combination thereof. In certain embodiments, the methods described herein further comprise co-administering an effective amount of ketamine by intravenous continuous infusion or intravenous bolus injection.

In certain embodiments, the methods described herein can be used to block or reduce sympathetic stimulation of one or more brain regions associated with anxiety disorders. In certain embodiments, the one or more brain regions associated with anxiety disorders can include one or more of the amygdala, the hippocampus, the insula, and the dorsal anterior cingulate cortex.

In certain embodiments, the methods described herein include administering a composition comprising an effective amount of bupivacaine multivesicular liposomes (MVLs) to one or more nerves of stellate ganglion of the patient, or an autonomic tissue area peripheral to the stellate ganglion, or a combination thereof.

In certain embodiments, stimulation of brain regions associated with anxiety disorders can be affected by the paravertebral chain such as for example, the C6, C7, C8, T1, and T2 regions (for example, the C6, C7, C8, T1, and T2 paravertebral ganglia) of the paravertebral chain. In some patients, the C6, C7, C8, T1, and T2 paravertebral ganglia are fused to form the stellate ganglion. Stimulation of brain regions associated with anxiety disorders may also be affected by several other regions of the sympathetic nervous system peripheral to the stellate ganglion. For example, in some patients, the T2 paravertebral ganglia is not part of the stellate ganglion. In some embodiments, the methods described herein can be employed on the one or more autonomic tissue regions peripheral to the stellate ganglion, such as the T2 paravertebral ganglia in patients in which it does not form part of the stellate ganglion to block or reduce sympathetic stimulation of one or more brain regions associated with anxiety disorders. In some embodiments, the methods described herein can be employed on one or more of the T1 region of the paravertebral chain, the T2 region of the paravertebral chain, and the stellate ganglion in order to block or reduce sympathetic stimulation to one or more brain regions associated with anxiety disorders. In some embodiments, the systems and methods described herein can be used to perform cryogenic therapy on one or more of the T1 region of the paravertebral chain, the T2 region of the paravertebral chain, and the stellate ganglion in order to block or reduce sympathetic stimulation to one or more brain regions associated with anxiety disorders.

In some embodiments, the methods described herein can be used to reduce sympathetic stimulation to one or more brain regions associated with anxiety disorders or the ability of the sympathetic nervous system to stimulate one or more brain regions associated with anxiety disorders by at least 10%, 15%, 20%, 25%, 30%, 35%, 40%, 45%, 50%, 55%, 60%, 65%, 70%, 75%, 80%, 85%, or 90%, or any other suitable amount or degree, when compared to the administration of the same dose amount of bupivacaine in an immediate release composition. In some embodiments, the systems and methods described herein can be used to reduce sympathetic stimulation to one or more brain regions associated with anxiety disorders or the ability of the sympathetic nervous system to stimulate one or more brain regions associated with anxiety disorders by between 25% and 75%, between 35% and 65%, between 25% and 50%, between 50% and 75%, or any other suitable ranges. The one or more brain regions associated with anxiety disorders can include one or more of the amygdala, the hippocampus, the insula, and the dorsal anterior cingulate cortex.

In some embodiments, the methods described herein can be used to reduce innervation to one or more brain regions associated with anxiety disorders by at least 10%, 15%, 20%, 25%, 30%, 35%, 40%, 45%, 50%, 55%, 60%, 65%, 70%, 75%, 80%, 85%, or 90%, or any other suitable amount or degree, when compared to the administration of the same dose amount of bupivacaine in an immediate release composition. In some embodiments, the methods described herein can be used to reduce innervation to one or more brain regions associated with anxiety disorders by between 25% and 75%, between 35% and 65%, between 25% and 50%, between 50% and 75%, or any other suitable range. The one or more brain regions associated with anxiety disorders can include one or more of the amygdala, the hippocampus, the insula, and the dorsal anterior cingulate cortex.

In some embodiments, the methods described herein can be used to reduce the release of catecholamines to one or more brain regions associated with anxiety disorders or the ability of the sympathetic nervous system to release catecholamines to one or more brain regions associated with anxiety disorders by at least 10%, 15%, 20%, 25%, 30%, 35%, 40%, 45%, 50%, 55%, 60%, 65%, 70%, 75%, 80%, 85%, or 90%, or any other suitable amount, when compared to the administration of the same dose amount of bupivacaine in an immediate release composition. In some embodiments, the methods described herein can be used to reduce the release of catecholamines to one or more brain regions associated with anxiety disorders or the ability of the sympathetic nervous system to release catecholamines to the one or more brain regions associated with anxiety disorders by between 25% and 75%, between 35% and 65%, between 25% and 50%, between 50% and 75%, or any other suitable range. The catecholamines may include norepinephrine and/or epinephrine. The one or more brain regions associated with anxiety disorders can include one or more of the amygdala, the hippocampus, the insula, and the dorsal anterior cingulate cortex.

In some embodiments, the methods described herein can be used to cause axonotmesis of at least 10%, 15%, 20%, 25%, 30%, 35%, 40%, 45%, 50%, 55%, 60%, 65%, 70%, 75%, 80%, 85%, or 90% of the nerves that provide sympathetic stimulation to one or more brain regions associated with anxiety disorders, or any other suitable amount or degree, as compared to the amount or degree of axonotmesis of the nerves that provide sympathetic stimulation to the one or more brain regions associated with anxiety disorders when the administration of the same dose amount of bupivacaine is in an immediate release composition. In some embodiments, the methods described herein can be used to cause axonotmesis of between 25% and 75%, between 35% and 65%, between 25% and 50%, or between 50% and 75% of the nerves that provide sympathetic stimulation to one or more brain regions associated with anxiety disorders, or any other suitable range. The one or more brain regions associated with anxiety disorders can include one or more of the amygdala, the hippocampus, the insula, and the dorsal anterior cingulate cortex.

In some embodiments, the methods described herein can be used to cause axonotmesis of at least 10%, 15%, 20%, 25%, 30%, 35%, 40%, 45%, 50%, 55%, 60%, 65%, 70%, 75%, 80%, 85%, or 90% of the nerves of the stellate ganglion and/or autonomic tissue peripheral to the stellate ganglion, or any other suitable amount or degree, as compared to the amount or degree of axonotmesis of the nerves of the stellate ganglion and/or autonomic tissue peripheral to the stellate ganglion when the administration of the same dose amount of bupivacaine is in an immediate release composition. In some embodiments, the methods described herein can be used to cause axonotmesis of between 25% and 75%, between 35% and 65%, between 25% and 50%, or between 50% and 75% of the nerves of the stellate ganglion, or any other suitable range.

Disabling the nerves of the stellate ganglion and/or autonomic tissue peripheral to the stellate ganglion using the methods disclosed herein can provide for prophylactic treatment to prevent various disorders, such as anxiety disorders, by temporarily preventing the release of certain neurotransmitters. Disabling the nerves of the stellate ganglion and/or autonomic tissue peripheral to the stellate ganglion using the methods disclosed herein may act as an alternative to a local anesthetic stellate ganglion block.

In certain embodiments, disabling the nerves of the stellate ganglion and/or autonomic tissue peripheral to the stellate ganglion using the methods disclosed herein can be used to treat a patient who has suffered from or is at risk of suffering anxiety disorders such as generalized anxiety disorders, panic disorder, post-traumatic stress disorder (PTSD), phobia related disorder (e.g., social phobia), and obsessive-compulsive disorder.

The methods described herein can allow for both short-term and long-term therapy for anxiety disorders. In certain embodiments, the axonotmesis of the nerve(s) using the methods described herein can provide for relief for anxiety disorders for an extended period of time (for example, up to 2 weeks, 3 weeks, 1 month, 2 months, 3 months, 4 months, 5 months, or 6 months). In certain embodiments, the methods described herein can advantageously be used to remodel the nerve(s) of the stellate ganglion and/or autonomic tissue peripheral to the stellate ganglion. For example, certain embodiments can cause axonotmesis of the nerve(s) of the stellate ganglion and/or autonomic tissue peripheral to the stellate ganglion, and the axon and myelin sheath can regenerate post-treatment. In certain embodiments, the nerve(s) may regenerate with a desired change in structure and/or function. For example, in some embodiments, the nerves can regenerate with a modified structure and/or function such that the treated condition no longer occurs following regeneration. For example, in certain embodiments, following regeneration, innervation may be reduced so that the previous condition no longer occurs. In certain embodiments, the nerves may be regenerate with new sodium channels having improved anatomical structures and/or function.

In some embodiments, the methods described herein further comprise assessing or receiving baseline information on one or more tests selected from the group consisting of Posttraumatic Stress Disorder Checklist (PCL-5), Headache Impact Test (HIT-6), Hospital Anxiety and Depression Scale (HADS), Quality of Life after Brain Injury (QoLIBRI), (Sheehan Suicide Tracking Scale (S-STS), Central Sensitization Inventory (CSI), headache frequency, average and worst headache scores (0-10 numerical rating scale (NRS)), and average and worst overall pain (0-10 NRS scale). In further embodiments, the method results in at least a 6-point decrease on HIT-6 or 10-point decrease on PCL-5, optionally combined with a Patients' Global Impression of Change (PGIC score)≥4/7. In certain embodiments, the change in HIT-6, PCL-5 or PGIC score is determined 4, 6, or 8 weeks after the administration of the composition of bupivacaine MVLs.

Patients

In some embodiments, the present disclosure includes identifying or selecting a patient to benefit from the treatments with bupivacaine MVLs as described herein. In some embodiments, the patient suffers from an anxiety disorder or a traumatic brain injury (TBI). In further embodiments, the patient has suffered from or is at risk of suffering from anxiety disorder or TBI. In some embodiments, the patient is in need of treating or ameliorating an overactive or unbalanced sympathetic nervous system. In some embodiments, the patient is in need of treating or ameliorating an anxiety disorder or a traumatic brain injury associated with an overactive or unbalanced sympathetic nervous system. In further embodiments, the patient has suffered from or is at risk of suffering from an overactive sympathetic nervous system. In some embodiments, the patient is in need of reducing or interrupting sympathetic stimulation to one or more brain regions associated with an anxiety disorder.

In some embodiments, the patient is in need of reducing or interrupting sympathetic stimulation to the nervous system. In further embodiments, the patient has suffered from or is at risk of suffering from overactive or overstimulated sympathetic nervous system that are associated with one or more conditions of an anxiety disorder or a traumatic brain injury. In some embodiments, the anxiety disorder is generalized anxiety disorder, panic disorder, post-traumatic stress disorder, phobia related disorder, or obsessive-compulsive disorder, or combinations thereof. In some embodiments, the anxiety disorder is post-traumatic stress disorder.

In some embodiments, the patient has suffered from or is at risk of suffering from generalized anxiety disorder. In some embodiments, the patient has suffered from or is at risk of suffering from panic disorder. In some embodiments, the patient has suffered from or is at risk of suffering from post-traumatic stress disorder. In some embodiments, the patient has suffered from or is at risk of suffering from phobia related disorder. In some embodiments, the patient has suffered from or is at risk of suffering from obsessive-compulsive disorder.

In some embodiments, the patient who suffered from or is at risk of suffering from PTSD has also suffered from or is at risk of suffering from a traumatic brain injury. In some embodiments, the patient who suffered from or is at risk of suffering from a traumatic brain injury has also suffered from or is at risk of suffering from PTSD.

In some embodiments, the patient has suffered from a TBI. In further embodiments, the patient also suffers from post-traumatic stress disorder (PTSD).

Dosage

In some embodiments of the methods disclosed herein, the concentration of bupivacaine in the composition is from about 1 mg/mL to about 30 mg/mL. In some embodiments, the concentration of bupivacaine in the composition is from about 1 mg/mL to about 30 mg/mL, from about 2 mg/mL to about 25 mg/mL, from about 5 mg/mL to about 20 mg/mL, or from about 10 mg/mL to about 15 mg/mL. In some embodiments, the concentration of bupivacaine in the composition is from about 12 mg/mL to about 14 mg/mL. In some embodiments, the concentration of bupivacaine in the composition is about 13.3 mg/mL. In other embodiments, the concentration of bupivacaine in the composition is from about 18 mg/mL to about 22 mg/mL, or about 20 mg/mL.

In some embodiments of the methods disclosed herein, the effective amount of bupivacaine administered is about 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 210 mg, 220 mg, 230 mg, 240 mg, 250 mg, 260 mg, 270 mg, 280 mg, 290 mg or 300 mg, or a range defined by any two of the preceding values. In further embodiments, the effective amount of from about 10 mg to about 200 mg, from about 20 mg to about 150 mg, from 25 mg to about 100 mg, or from 30 mg to about 75 mg. In one embodiment, the amount of bupivacaine administered is about 66.5 mg. I another embodiment, the amount of bupivacaine administered is about 106 mg. In another embodiment, the amount of bupivacaine administered is about 133 mg.

In some embodiments of the methods disclosed herein, the total volume of the bupivacaine MVL composition administered is about 1 mL, 2 mL, 3 mL, 4 mL, 5 mL, 6 mL, 7 mL, 8 mL, 9 mL, 10 mL, 11 mL, 12 mL, 13 mL, 14 mL, 15 mL, 16 mL, 17 mL, 18 mL, 19 mL or 20 mL, or a range defined by any two of the preceding values. In further embodiments, the total volume of the bupivacaine MVL composition administered is from about 1 mL to about 20 mL, from about 2 mL to about 15 mL, from about 3 mL to about 10 mL or from about 4 mL to about 8 mL, or about 5 mL. The total volume refers to the volume administered in a single dose in one area, for example, either to the left stellate ganglion or the right stellate ganglion. For bilateral SGB, if the volume administered to each side is about 5 mL, then the patient receives a total volume of 10 mL. In further embodiments, the total volume of the composition administered is from about 5 mL to about 15 mL for unilateral stellate ganglion block. In another embodiment, the total volume of the bupivacaine MVLs composition administered is about 10 mL for unilateral stellate ganglion block. In further embodiments, the total volume of the composition administered is from about 10 mL to about 20 mL for bilateral stellate ganglion block. In one embodiment, the total volume of the composition administered is about 20 mL for bilateral stellate ganglion block.

In any embodiments of the method described herein, the composition of bupivacaine MVLs may extend the effect or duration of the SGB by at least 10%, 15%, 20%, 25%, 30%, 35%, 40%, 45%, 50%, 60%, 70%, 80%, 90%, or 100%, when compared to the administration of the same dose amount of bupivacaine in an immediate release composition.

In some embodiments of the methods disclosed herein, the amount of midazolam administered is about 1 mg, 1.1 mg, 1.2 mg, 1.3 mg, 1.4 mg, 1.5 mg, 1.6 mg, 1.7 mg, 1.8 mg, 1.9 mg, 2 mg, 2.1 mg, 2.2 mg, 2.3 mg, 2.4 mg, 2.5 mg, 2.6 mg, 2.7 mg, 2.8 mg, 2.9 mg, 3 mg, 3.1 mg, 3.2 mg, 3.3 mg, 3.4 mg, 3.5 mg, 3.6 mg, 3.7 mg, 3.8 mg, 3.9 mg, or 4 mg, or a range defined by any two of the preceding values. In further embodiments, the amount of midazolam administered is from about 1 mg to about 4 mg, or about 2 mg.

In some embodiments of the methods disclosed herein, the total effective amount of ketamine administered is about 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, or 1 mg/kg, or a range defined by any two of the preceding values. In further embodiments, the total effective amount of ketamine administered is from about 0.1 mg/kg to about 1 mg/kg, from about 0.1 mg/kg to about 0.3 mg/kg, or from about 0.4 mg/kg to about 1 mg/kg. In some embodiments, the amount of time of continuous infusion or intravenous bolus injection of ketamine is about 30 minutes, 45 minutes, or 60 minutes, or a range defined by any two of the preceding values. For example, in some embodiments, the amount of time of continuous infusion or intravenous bolus injection of ketamine is about 30-45 minutes.

### Methods of Administration

Some embodiments of the present application are related to methods of administering a composition comprising an effective amount of bupivacaine MVLs, as described herein, to one or more nerves of stellate ganglion of a patient, or an autonomic tissue area peripheral to the stellate ganglion. In some embodiments, a treatment apparatus comprises a needle and a compartment for the bupivacaine MVLs.

In some embodiments of the methods described herein, the administration includes navigating a treatment apparatus to stellate ganglion of the patient, or an autonomic tissue area peripheral to the stellate ganglion; injecting bupivacaine encapsulated MVLs to one or more nerves of the stellate ganglion, or to one or more nerves of the autonomic tissue area peripheral to the stellate ganglion, or both; and removing the treatment apparatus from the patient.

In some embodiments of the method described herein, navigating the treatment apparatus to the stellate ganglion includes inserting the needle of the treatment apparatus percutaneously to one or more nerves of the stellate ganglion area, or to one or more nerves of the autonomic tissue area peripheral to the stellate ganglion, or both. For example, through an anterior wall of the trachea to tissue comprising a deep cardiac plexus. In some embodiments, navigating the treatment apparatus to the stellate ganglion comprises placing the needle above the longus colli muscle. In some embodiments, navigating the treatment apparatus to the stellate ganglion includes guiding the needle using an imaging guide. In further embodiments, the imaging guide comprises fluoroscopic or ultrasound imaging guide. The patient may be under general anesthesia or be intubated. In some embodiments, the administration is to the left stellate ganglion. In some embodiments, the administration is to the right stellate ganglion. In some embodiments, the administration is to both the left and the right stellate ganglion.

In some embodiments, the bupivacaine MVLs may be administered by bolus injection, continuous infusion, or a combination thereof. Administration of the instant bupivacaine MVL composition may also be accomplished using standard methods and devices, e.g., injector systems, needle and syringe, a subcutaneous injection port delivery system, and the like. The administration of the bupivacaine MVLs composition may be used in conjunction with Pacira's handheld cryoanalgesia device Iovera®.

### EXAMPLES

The following examples, including experiments and results achieved, are provided for illustrative purposes only and are not to be construed as limiting the present application.

### Example 1: Stellate Ganglion Blockade Procedure

In this example, patients suffering from or at risk of suffering from PTSD are selected. Bupivacaine encapsulated multivesicular liposomes (e.g., Exparel®) is injected anterior to the patient's longus colli muscle. Ultrasound guidance is used to introduce a 2.5 cm 25-gauge needle at the patient's C6 level for continuous infusion of bupivacaine encapsulated MVLs of the present disclosure. The mean infusion rate is about 5 mL per hour. The needle is directed toward the longus colli muscle overlying Chassaignac's tubercle, following an oblique path medial to the common carotid artery and jugular vein.

While the present application has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

### Example 2: Treatment of Headaches Associated with Traumatic Brain Injury

A multicenter randomized, double-blind, placebo-controlled, parallel-group study to evaluate the efficacious treatments of ketamine and stellate ganglion block (SGB) with bupivacaine infusion, compared to sham therapies, for headaches associated with traumatic brain injury (TBI) and post-traumatic stress disorder (PTSD) is summarized herein.

Eligible subjects will be randomized into one of four groups, Groups A, B, C, and D, in a 2:2:2:1 ratio, respectively. Subjects in Groups A, B, and C will receive at least one intervention, while subjects in Group D will receive sham SGB and serve as a comparator. A summary of the treatment design for each of the groups is summarized below:
Group A: Subjects will receive 8 mL of 0.25% bupivacaine SGB and placebo ketamine. The placebo ketamine group will consist of a 1-4 mg midazolam bolus before the procedure (procedural sedation) followed by a continuous infusion or IV boluses over 30-45 minutes of 1-7 mg midazolam (active placebo) titrated to effect. The technical success of the block will be confirmed by pre- and post-injection temperature measurements on the palmar surface of the fingertips.
Group B: Subjects will receive a sham SGB and a ketamine infusion. For the sham SGB, 2 mL of saline will be administered into the subcutaneous tissues in the neck under image guidance. Prior to the procedure, patients will receive 1-4 mg midazolam and up to 0.3 mg/kg ketamine. After the procedure, patients will receive intravenous ketamine (IV boluses or infusion) for 30-45 minutes for a total dose of 0.5-1 mg/kg. The administration times and doses will be based on the patient responses and clinical judgment of the anesthesiologist, consistent with personalized medicine. If needed, higher doses of midazolam may be given as needed to prevent side effects.
Group C: Subjects will receive the SGB as outlined for Group A and the ketamine infusion as outlined for Group B.
Group D: Subjects will receive a sham SGB as outlined for Group B and a placebo ketamine infusion (midazolam) as outlined for Group A.

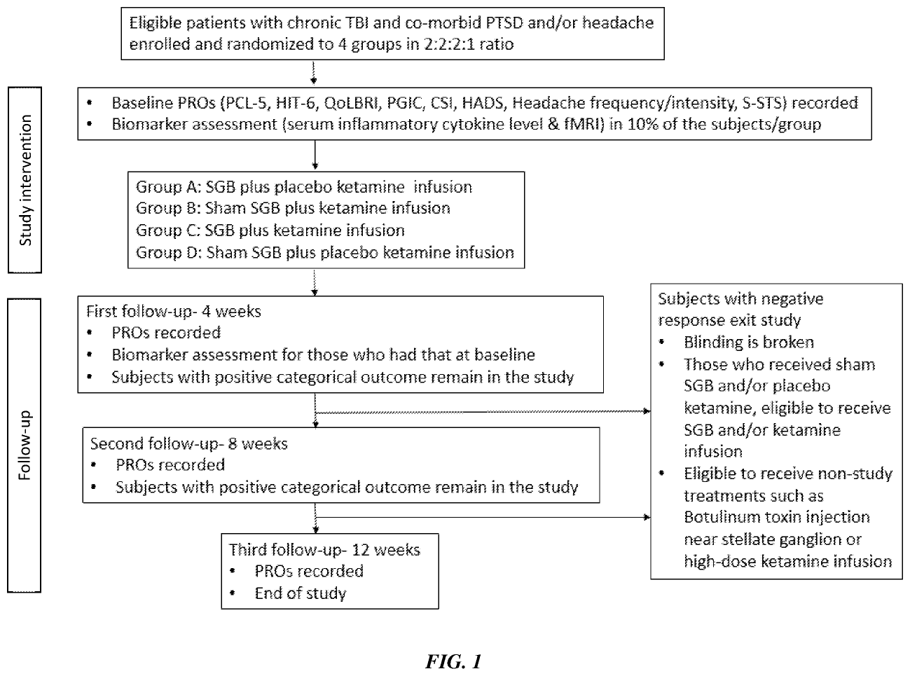

Several patient-reported outcomes, including quality of life measures, will be collected at baseline and the primary endpoint at 4 weeks. Those with a positive categorical response will be followed further at 8 and 12 weeks. Those with negative outcomes will exit the study and be followed as an observational cohort where they will be eligible for non-study measures as determined by the treating providers. This may include other novel treatments such as using higher doses of ketamine, sympathetic blocks with botulinum toxin, and the use of neuromodulation. The overall study is summarized in FIG. 1.

### Study Interventions

All participants will receive a peripheral intravenous (IV) catheter prior to the procedure, which is standard of care for SGB and necessary for IV ketamine. The patient will be placed in the supine position. Monitors, including a blood pressure cuff and pulse oximeter, will be used to monitor vital signs. A fluid bolus will be ready for administration by the nursing staff via peripheral IV in case of hypotension along with standard resuscitative equipment. Patients will receive midazolam for pre-procedural sedation. The ketamine group will receive 0.3 mg/kg ketamine bolus, in addition to midazolam, as pre-procedural sedation. Test medication infusion or small boluses (ketamine 0.5-1 mg/kg in 100 ml of normal saline or 10-20 mg bolus doses, or active placebo-midazolam 1-7 mg) will be started after the procedure and titrated to effect by the clinician or a trained anesthesiologists. Both midazolam and ketamine are clear solutions and not distinguishable, which will facilitate blinding.

Stellate Ganglion Blocks and Subgroup Allocations

The SGB will be administered on the right stellate ganglion of the subjects. However, left-handed or ambidextrous patients who do not respond to right-sided SGBs may be eligible to receive left-side SGBs outside of the controlled trial. Subjects receiving an SGB will be suballocate via a random number table to receive conventional immediate release bupivacaine or liposomal bupivacaine (e.g., Exparel®) to evaluate whether the longer transit time and acute pain relief experienced with liposomal bupivacaine translates to longer relief of PTSD and post-traumatic headache symptoms. Without being bound by a particular theory, the sustained release of liposomal bupivacaine may provide an enduring treatment for post-traumatic headaches and PTSD symptoms.

Patients' necks will be prepped and draped in a sterile fashion and landmarks identified under fluoroscopy or ultrasonography (USG). Subcutaneous local infiltration will be done with 0.5 to 3 mL of 1% lidocaine using a 25- to 30-gauge needle. For SGB, a longer (2-4 inches) 21- to 25-gauge needle will be placed above the longus colli muscle just medial to C6 or C7 transverse process under image guidance. In the sham block group, the needle will be placed superficially to mitigate risk (e.g., vascular compromise). Contrast injection will be utilized for procedures done under fluoroscopic guidance to rule out vascular uptake and ensure adequate injectate spread. For SGB, 5-10 mL of 0.25% bupivacaine will be injected, depending on contrast spread. For sham SGB, 2 mL of normal saline will be injected into the subcutaneous tissue.

Multiple pain, functional, and psychometric assessments, including quality of life assessments, will be done at baseline and at the 4-week follow-up visit (primary outcome). Those with positive categorical outcomes will continue to be followed at 8 and 12 weeks with a similar battery of assessments. A positive categorical outcome is defined as at least a 6-point decrease on HIT-6 or 10-point decrease on PCL-5 combined with a PLIC score of 4/7 or greater. Those with negative categorical outcomes at 4, 8 or 12 weeks will exit the study, and blinding will be broken at this stage.

### Inclusion/Exclusion Criteria

Inclusion Criteria

The following is a list of inclusion criteria for subjects:
Adults 18 years or older
Mild or moderate/severe TBI (defined by the U.S. Departments of Defense as a head injury with loss of consciousness (LOC) of 30 minutes or more, alteration of consciousness (AOC) or post-traumatic amnesia (PTA) of 24 hours or more, or radiological findings of intracranial abnormalities secondary to head trauma). Participants will be categorized as having mild TBI if they did not meet the criteria for moderate-to-severe TBI but had a head injury with positive LOC and/or PTSD (score of 30 or more on PCL-5).
   If TBI, history of headache or occipital pain (average pain score of 4/10 in the past week)
Duration of chronic TBI or PTSD of 3 months or more

Exclusion Criteria

The following is a list of exclusion criteria for subjects:
Ketamine infusion or SGB within the past 6 months
Serious medical or psychiatric conditions other than TBI or PTSD that could affect cognition (e.g., dementia, Parkinson's Disease)
Elevated intracranial pressure
Active psychosis or poorly controlled non-injury or PTSD-related psychiatric condition (e.g., bipolar disorder)
Poorly controlled medical conditions that could be exacerbated by treatment (e.g., unstable angina)

Pain, Functional, and Psychometric Assessments

Primary outcomes include measures of Headache Impact Test (HIT-6) and PTSD Checklist (PCL-5). HIT-6 is a reliable 6-item instrument that measures the impact of headaches in a 1-month period that has been validated for a variety of headache conditions, including tension-type headache and migraine. PCL-5 is a psychometrically sound 20 item instrument that assesses 20 DSM-5 symptoms of PTSD and has been shown to have high internal consistency and test-retest reliability in Veteran and military service members.

Secondary outcomes include measurements of the following eight factors:
1. Quality of Life after Brain Injury (QoLIBRI): The QoLIBRI is a health-related quality of life scale that was specifically developed for TBI and can detect the effects of interventions. It measures physical, psychological, daily life, and psychosocial changes typical of TBI and consists of a total of 37 questions in 6 categories, rated on 5-point Likert scales. The subscale scores can be used separately, and responses can be summed to give a total score. The first four categories assess ‘satisfaction’ while the last two categories assess ‘feeling bothered’ with key aspects of life.
2. Patient global impression of change (7-point Likert scale) which represents the subject's overall belief about the effectiveness of treatment on a 7-point categorical rating scale and identifies clinically significant changes in a patient's condition.
3. Central sensitization inventory (CSI) is a 25-item measure of central sensitization. It is a self-reported tool that consists of statements related to current health symptoms rated on a 5-point Likert scale, with a score of 40 being the cut off value for central sensitization. It has been found to be responsive to treatment outcomes.
4. Headache intensity (average and worst pain)—average and worst headache scores (0-10 numerical rating scale (NRS)), average and worst overall pain (0-10 NRS scale), recorded over the week before the visit.
5. Headache frequency in the past week—days per week with scores greater or equal to 2, and 7/10, recorded over the past week.
6. Hospital Anxiety and Depression Scale (HADS) is a simple, easy-to-use tool that assesses both anxiety and depression.
7. Sheehan Suicidality Tracking Scale (S-STS) is a short scale designed to assess and monitor suicidality over time and is very sensitive to changes in treatment outcomes, which will be tracked in subjects who score >4 on their initial intake.
8. Analgesic or psychotropic medication reduction—defined as >20% reduction in opioid, barbiturate or benzodiazepine, or cessation, cessation of nonopioid analgesic, or >50% reduction in non-benzodiazepine sedation or psychotropic medication (e.g., antidepressant or antipsychotic).

In addition, to assess for any changes in inflammatory markers with study treatments, biomarker measurement will be collected at baseline and at 4-week follow-up visits for up to 10% in each group as available at individual sites in which this is available. For example, inflammatory markers in the blood will be collected for individuals with PTSD and pain exhibit significantly elevated levels of proinflammatory markers, such as interleukin-1β (IL-1β), interleukin-6 (IL-6), tumor necrosis factor-α (TNFα), neurofilament light chain (NfL), glial fibrillary acidic protein (GFAP), C-reactive protein, nerve growth factor (NGF) and brain derived neurotrophic factor (BDNF).

### MRI Analysis

All MRI analyses will be performed by a person blinded to clinical outcomes to prevent bias in quality control decisions or analyses. Given that these analyses will be conducted in 10% of the study sample, they are intended to be exploratory.

Structural MRI Analysis: T1-weighted structural images will be segmented and processed for voxel-wise based morphology using the diffeomorphic anatomical registration through exponentiated lie algebra (DARTEL) toolbox in SPM. Images will then be normalized, modulated, and smoothed. Statistically significant regions will be extracted from the normalized, smoothed images and further analyzed in SPSS. Areas of interest include various cortical (somatosensory, insular, cingulate and prefrontal cortices, and precuneus) and subcortical (nucleus accumbens, amygdala, thalamus, periaqueductal gray, and rostral ventral medulla) regions.

Resting fMRI Analysis: The seeds of interest include areas of DMN (medial prefrontal cortex and precuneus), descending antinociceptive pathway (rostral ventral medulla and periaqueductal gray), and regions known to have an abundance of NMDA receptors (amygdala). The average time course of seed regions (3 mm radius spheres) will be used as regressors in a whole-brain GLM to find other brain regions whose activity correlates with that of the seed. The resulting single subject maps will be normalized to standard space and passed up to group and correlation analyses.

1. A method of treating a patient who has suffered from or is at risk of suffering from an anxiety disorder or a traumatic brain injury (TBI), comprising:
administering a composition comprising an effective amount of bupivacaine multivesicular liposomes (MVLs) to one or more nerves of the stellate ganglion of the patient, or an autonomic tissue area peripheral to the stellate ganglion, or both.

2. (canceled)

3. A method of treating or ameliorating an anxiety disorder or a traumatic brain injury associated with an overactive or unbalanced sympathetic nervous system in a patient in need thereof, comprising:
administering a composition comprising an effective amount of bupivacaine MVLs to one or more nerves of the stellate ganglion of the patient, or an autonomic tissue area peripheral to the stellate ganglion, or both.

4. (canceled)

5. (canceled)

6. (canceled)

7. The method of claim 1, wherein the anxiety disorder comprises generalized anxiety disorder, panic disorder, post-traumatic stress disorder (PTSD), or phobia related disorder.

8. (canceled)

9. The method of claim 7, wherein the patient who suffered from or is at risk of suffering from PTSD has also suffered from or is at risk of suffering from a traumatic brain injury, or wherein the patient who suffered from or is at risk of suffering from a traumatic brain injury has also suffered from or is at risk of suffering from PTSD.

10. (canceled)

11. The method of claim 1, wherein the one or more nerves of the stellate ganglion comprise one or more nerves that provide sympathetic stimulation to one or more brain regions associated with the anxiety disorder.

12. A method of treating or ameliorating one or more symptoms associated with a traumatic brain injury (TBI) in a patient in need thereof, comprising:
identifying or selecting a patient who has suffered from a TBI;
administering a composition comprising an effective amount of bupivacaine multivesicular liposomes (MVLs) to one or more nerves of the stellate ganglion of the patient, or an autonomic tissue area peripheral to the stellate ganglion, or both.

13. (canceled)

14. (canceled)

15. (canceled)

16. The method of claim 1, further comprising measuring or receiving information on a baseline level of one or more proinflammatory biomarkers in the blood prior to the administration of the composition of bupivacaine MVLs.

17. The method of claim 16, wherein the one or more inflammatory biomarkers are selected from the group consisting of interleukin-1β (IL-1β), interleukin-6 (IL-6), tumor necrosis factor-α (TNFα), neurofilament light chain (NfL), glial fibrillary acidic protein (GFAP), C-reactive protein, nerve growth factor (NGF) and brain derived neurotrophic factor (BDNF).

18. The method of claim 16, wherein the method reduces the level of one or more proinflammatory biomarkers in the blood.

19. (canceled)

20. The method of claim 1, further comprising co-administering an effective amount of ketamine by intravenous continuous infusion or intravenous bolus injection.

21. The method of claim 20, wherein the effective amount of ketamine is from about 0.5 to about 1 mg/kg.

22. The method of claim 1, wherein the method causes a temporary reduction in the ability of the sympathetic nervous system to release norepinephrine.

23. (canceled)

24. The method of claim 1, wherein the method results in at least a 6-point decrease on Headache Impact Test (HIT-6) or 10-point decrease on Posttraumatic Stress Disorder Checklist (PCL 5), optionally combined with a Patients' Global Impression of Change (PGIC score)≥4/7.

25. The method of claim 24, wherein the change in HIT-6, PCL-5 or PGIC score is determined 4, 6, or 8 weeks after the administration of the composition of bupivacaine MVLs.

26. The method of claim 1, wherein the concentration of bupivacaine in the composition is from about 1 mg/mL to about 30 mg/mL, or about 13.3 mg/mL.

27. (canceled)

28. The method of claim 1, wherein the effective amount of bupivacaine administered is from about 10 mg to about 200 mg, from about 20 mg to about 100 mg, or from about 25 mg to about 75 mg.

29. The method of claim 1, wherein the total volume of the composition administered is from about 5 mL to about 10 mL for unilateral stellate ganglion block, or about 10 mL to about 20 mL for bilateral stellate ganglion block.

30. (canceled)

31. (canceled)

32. (canceled)

33. The method of claim 1, wherein the bupivacaine is in the form of bupivacaine phosphate.

34. The method of claim 1, wherein the administering temporarily reduces or interrupts sympathetic stimulation to the nervous system for up to 3 days, 7 days, 10 days, 14 days, 21 days, 1 month, 2 months, 3 months, 4 months, 5 months or 6 months.

35.-44. (canceled)

45. The method of claim 1, wherein the one or more desired nerves of the stellate ganglion or the autonomic tissue area peripheral to the stellate ganglion comprise one or more desired nerves of the stellate ganglion or one or more desired nerves of the autonomic tissue area peripheral to the stellate ganglion.

46. (canceled)

47. The method of claim 1, wherein the one or more desired nerves of the stellate ganglion or the autonomic tissue area peripheral to the stellate ganglion comprise one or more desired nerves of a paravertebral chain.

48. The method of claim 47, wherein the one or more desired nerves of the paravertebral chain comprises one or more desired nerves of one or more of a C7 region of the paravertebral chain, a C8 region of the paravertebral chain, a T1 region of the paravertebral chain, or a T2 region of the paravertebral chain, or combinations thereof, or wherein the one or more desired nerves of the paravertebral chain comprise one or more desired nerves of the T1 region of a paravertebral chain or the T2 region of the paravertebral chain, or a combination thereof, or wherein the one or more desired nerves of the paravertebral chain comprises one or more desired nerves of one or more of a C6 region of the paravertebral chain, a C7 region of the paravertebral chain, or combinations thereof.

49. (canceled)

50. (canceled)
